(19) United States
(12) Patent Application Publication
(10) Pub. No.: US 2026/0083481 A1
(43) Pub. Date: Mar. 26, 2026

(54) ROD COUPLING ASSEMBLIES FOR BONE STABILIZATION CONSTRUCTS

(71) Applicant: SI-Bone Inc., Santa Clara, CA (US)

(72) Inventors: Francois FOLLINI, Austin, TX (US); Derek P. LINDSEY, San Jose, CA (US); Bret SCHNEIDER, San Jose, CA (US)

(21) Appl. No.: 19/303,255

(22) Filed: Aug. 18, 2025

Related U.S. Application Data
(63) Continuation of application No. 17/777,679, now Pat. No. 12,419,668.
(60) Provisional application No. 62/938,546, filed on Nov. 21, 2019.

(51) Int. Cl. A61B 17/70

(52) U.S. Cl. CPC A61B 17/7037; A61B 17/7032; A61B 17/7098

(57) ABSTRACT

Bone implants and stabilizing rod coupling assemblies to secure a stabilizing rod relative to the bone implant. Bone implants may include a radially recessed collet receiving region that includes a proximal end with an undercut ledge configuration that is sized and configured to interface with and couple to a collet of a tulip assembly.

### CROSS REFERENCE TO RELATED APPLICATIONS

This application is a continuation of U.S. patent application Ser. No. 17/777,679, filed May 18, 2022, which is a 371 of International Application No. PCT/US2020/061849, filed Nov. 23, 2020, which claims the benefit of U.S. Provisional Application No. 62/938,546, filed Nov. 21, 2019, the entire disclosures of which are incorporated by reference herein for all purposes.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Bone implants may be used to assist in the fixation of bones in spine surgeries such as, but not limited to, spinal fusions. Proximal regions of the bone implants may be configured to be coupled to a tulip. The tulip is generally rotatable relative to the implant in a first moveable state, the tulip being further configured to accept a rod for fixation.

Implants, tulips, and assemblies are generally needed to securely couple the implant, tulip and the rod.

### SUMMARY OF THE DISCLOSURE

One aspect of the disclosure is a rod coupling system. The rod coupling system may include a bone implant and a tulip assembly.

In this aspect, the bone implant may have a proximal region spaced from a distal region, the proximal region sized and configured to interface with a tulip assembly. The tulip assembly may include a tulip and a collet sized and configured to be disposed at least partially within the tulip. The bone implant proximal region may include a radially recessed collet receiving region configured and sized to receive therein a radially inward protruding implant interface region of the collet, the recessed collet receiving region including a radially extending surface that is disposed at an angle less than ninety degrees relative to a bone implant longitudinal axis, the angle measured distally relative to the radially extending surface. The tulip assembly may have an opening therethrough sized and configured to receive a stabilizing rod therethrough. The implant interface region of the collet may include a radially extending surface disposed at an angle relative to a collet longitudinal axis, the radially extending surface of the implant interface region positioned to be facing the radially extending surface of the recessed collet receiving region when the implant interface region is interfaced with the recessed collet receiving region.

In this aspect, the radially extending surface of the implant interface region of the collet may be orthogonal to the longitudinal axis of the collet.

In this aspect, the radially extending surface of the recessed collet receiving region may be disposed at an angle less than 85 degrees relative to the longitudinal axis of the implant.

In this aspect, the radially extending surface of the recessed collet receiving region may be disposed at an angle greater than 45 degrees relative to the longitudinal axis of the implant.

In this aspect, the radially extending surface of the recessed collet receiving region may be disposed at an angle greater than 75 degrees relative to the longitudinal axis of the implant.

In this aspect, the radially extending surface of the recessed collet receiving region may be disposed at an angle from seventy degrees to eighty-nine degrees relative to the longitudinal axis of the implant.

In this aspect, the tulip assembly may optionally be modular such that it may be adapted to be delivered separately from the bone implant and movably secured to the bone implant after the bone implant has been at least partially implanted within bone. The implant proximal end may include an internal thread sized and configured to receive therein a threaded guide rod, the tulip assembly adapted to be advanced over the threaded guide rod and advanced over the proximal end of the implant to couple the tulip assembly and the bone implant.

In this aspect, the bone implant proximal region may include a chamfered proximal end to facilitate expansion of a collet distal end as the collet is advanced over the chamfered proximal end, the recessed collet receiving region causing the radially inward protruding implant interface region of the collet to move radially inward into the recessed collet receiving region as the collet is advanced distally over the bone implant proximal region. Coupling the collet and implant may occur before the implant is implanted into bone.

In this aspect, the bone implant proximal region may be configured to facilitate opening of the collet distal end as the collet is advanced over the proximal end, the recessed collet receiving region causing the radially inward protruding implant interface region of the collet to revert towards an at-rest state into the recessed collet receiving region as the collet is advanced further over the bone implant.

In this aspect, the recessed collet receiving region may have a depth from 0.1 mm to 2.0 mm.

In this aspect, the bone implant proximal region may be void of an external thread.

In this aspect, the bone implant proximal region may have an outermost sleeve surface that is sized and configured to extend over at least a portion of a distal end of the tulip. An outermost sleeve surface may be a surface of an outer implant sleeve, the outer implant sleeve disposed about an implant inner shank.

In this aspect, the recessed collet receiving region may have an annular configuration about the bone implant.

One aspect of this disclosure is a bone implant with a recessed collet receiving region, the recessed collet receiving region including a radially extending surface that is disposed at an angle less than ninety degrees relative to a bone implant longitudinal axis, the angle measured distally relative to the radially extending surface, the radially extending surface angled so as to reduce the likelihood of the collet expanding and becoming loose in response to an axial force compared to a system alike in all ways but instead having a radially extending surface that is orthogonal to the bone implant longitudinal axis.

This aspect may include any other suitable features of bone implants, systems or assemblies herein.

One aspect of this disclosure is a tulip assembly for securing to a bone implant and to a stabilizing rod. The tulip assembly may be modular or not modular.

In this aspect, the tulip may include one or more side openings that are sized and configured to receive an elongate stabilizing rod therethrough.

This aspect may include a collet with a partially spherical configuration, the collet sized to be disposed at least partially within the tulip, the tulip having an inner surface and the collet having an outer surface that are together sized and configured to interface with each other when the collet is disposed within the tulip, the tulip inner surface and the collet outer surface both configured relative to each other to cause the tulip to be movable relative to the collet in a movable arrangement.

In this aspect, the collet may include a radially inward protruding implant interface region, the protruding implant interface region including a radially extending surface disposed at an angle relative to a collet longitudinal axis and an axially extending surface that meets the radially extending surface, the radially inward protrusion facilitating a collet distal end to collapse towards a recessed region in a bone implant proximal region as the collet is advanced over the proximal region of the bone implant.

In this aspect, the tulip assembly may include a saddle sized and configured to be disposed within the tulip and at least partially proximal to the collet, the saddle having a distal region with an inner curved surface shaped to mate with a curved proximal region of the collect, the saddle having a proximal end with a rod recessed region with a configuration shaped to interface with the elongate rod.

In this aspect, a collet radially inward protrusion may have a height of 0.1 mm to 2.0 mm.

In this aspect, the radially extending surface may be orthogonal to a collet longitudinal axis.

In this aspect, the axially extending surface may be parallel to a collet longitudinal axis.

In this aspect, the axially extending surface may be tapered and may be disposed at an angle between zero and twenty degrees relative to a collet longitudinal axis.

One aspect of the disclosure is a bone implant adapted to be coupled to a tulip assembly. The bone implant has an elongate body with a long axis and a proximal region, wherein the proximal region sized and configured to be coupled within a collet of a tulip assembly. In this aspect, the proximal region may include a radially recessed collet receiving region including a radially extending surface that is disposed at an angle less than ninety degrees relative to the long axis, a distal axially extending surface and a proximal axially extending surface, the distal and proximal axially extending surfaces on either side of the radially extending surface, the proximally axially extending surface disposed further radially outward from the long axis than the distal axially extending surface.

In this aspect, a proximal end of the proximal region may have a chamfered configuration.

In this aspect, the distal axially extending surface may be parallel with the long axis.

In this aspect, the distal axially extending surface may be at an angle from zero to twenty-five degrees relative to the long axis.

In this aspect, the proximally axially extending surface may be parallel with the long axis.

In this aspect, the elongate body proximal region may be void of an outer thread.

In this aspect, the radially recessed collet receiving region may have an annular configuration about the proximal region of the elongate body.

In this aspect, the radially recessed collet receiving region may have a depth from 0.1 mm to 2.0 mm relative to an outer dimension of the implant axially adjacent to the recessed collet receiving region.

One aspect of this disclosure is a bone implant that is adapted to be coupled to a tulip assembly. The bone implant may have a long axis and a proximal region, wherein the proximal region is sized and configured to be coupled to a collet of a tulip assembly. The proximal region may include a radially recessed collet receiving area having a proximal end with an undercut ledge.

This aspect may include any other suitable feature of any implant, system or assembly herein.

### BRIEF DESCRIPTION OF THE DRAWINGS

FIG. 1A illustrates an exemplary rod coupling system in an exemplary stabilized state.

FIGS. 1B, 1C, 1D and 1E are sectional views showing an exemplary rod stabilizing sequence of steps.

FIG. 2A illustrates an exploded view of an exemplary rod coupling system.

FIG. 2B illustrates an exemplary rod coupling system with the implant uncoupled from a tulip assembly.

FIG. 2C illustrates a sectional view of an exemplary rod coupling system with the implant uncoupled from a tulip assembly.

FIG. 2D illustrates a sectional view of a portion of an exemplary tulip assembly.

FIG. 2E illustrates an exemplary rod coupling system with the implant coupled with a tulip assembly.

FIG. 2F illustrates a sectional view of an exemplary rod coupling system with the implant coupled to a tulip assembly.

FIG. 2G illustrates a sectional view of a portion of an exemplary tulip assembly interfacing a proximal region of a bone implant.

FIG. 2H illustrates an exemplary rod coupling system with the implant coupled and fixedly secured with a tulip assembly.

FIG. 2I illustrates a sectional view of an exemplary rod coupling system with the implant coupled and fixedly secured to a tulip assembly.

FIG. 2J illustrates a sectional view of a portion of an exemplary tulip assembly interfacing and fixedly secured with a proximal region of a bone implant.

FIGS. 3A and 3B illustrate a rod coupling assembly interfaced with a threaded tool that may be used for removal.

FIG. 4A illustrates an exploded view of an exemplary rod coupling system.

FIG. 4B illustrates an exemplary rod coupling system with the implant uncoupled from a tulip assembly.

FIG. 4C illustrates a sectional view of an exemplary rod coupling system with the implant uncoupled from a tulip assembly.

FIG. 4D illustrates an exemplary rod coupling system with the implant coupled with a tulip assembly.

FIGS. 4E, 4F, 4G and 4H illustrate a rod coupling system with split annular member configured to interface with a bone implant.

FIG. 5A illustrates an exemplary rod coupling system with the implant uncoupled from a tulip assembly.

FIGS. 5B, 5C and 5D illustrates an exemplary rod coupling system with the implant coupled to a tulip assembly, with a split annular member part of the tulip assembly.

FIG. 6A illustrates an exploded view of an exemplary rod coupling system.

FIG. 6B illustrates an exemplary rod coupling system with the implant uncoupled from a tulip assembly.

FIG. 6C illustrates a sectional view of an exemplary rod coupling system with the implant uncoupled from a tulip assembly.

FIG. 6D illustrates an exploded view of an exemplary rod coupling system.

FIG. 6E is a perspective view of an exemplary rod coupling system with the implant coupled to a tulip assembly.

FIGS. 7A, 7B, 7C, 7D and 7E illustrate an exemplary embodiment in which a proximal portion of the implant is sized and configured relative to a tulip to interface with a surface of the tulip of the tulip assembly.

FIGS. 8A, 8B, 8C, 8D, 8E, 8F and 8G illustrate an exemplary embodiment in which a proximal portion of the implant is sized and configured relative to a tulip to interface with a surface of the tulip of the tulip assembly.

FIGS. 9A, 9B, 9C, 9D, 9E and 9F illustrate an example of a tulip assembly that includes an inner tulip member and an outer tulip member.

FIGS. 10A, 10B, 10C, 10D, 10E, 10F, 10G, 10H and 10I illustrate an example of a tulip assembly that includes an inner tulip member and an outer tulip member.

FIGS. 11A, 11B, 11C, 11D, 11E, 11F, 11G, 11H, 11I and 11J are an example of a rod coupling assembly in which an implant proximal end includes a collet receiving region with a proximal end having an undercut ledge configuration.

### DETAILED DESCRIPTION

The disclosure herein is related to rod coupling systems or assemblies. The rod coupling assemblies herein include a tulip, and wherein the assemblies are adapted and configured to be coupled to a stabilizing rod that is part of a bone stabilization construct. The rod coupling assemblies in this context may also be considered to include an implant, or have an implantable portion, that is secured in tissue (e.g. bone), wherein the implant is coupled to the tulip. The rod may be part of a larger construct that may be used to stabilize one or more regions of a patient's body, such as a spine or pelvis. In some embodiments, the rod coupling assemblies herein may be disposed in a lower region of the spine, such in the region of a sacro-iliac joint.

The disclosure herein that is related to the rod coupling assemblies is related to some aspects of the disclosure in published application WO2020/168269, which is fully incorporated by reference herein for all purposes. For example, and without limitation, FIGS. 25A-26B in publication WO2020/168269 show an exemplary rod coupling assembly that includes a head portion 2506 that may be provided with a coupler 2524 and a main body 2526 and a nut (not shown). The nut may have external threads that mate with internal threads located in the proximal recess of main body 2526 to tighten a spinal rod (not shown) against the bottom of channels 2528 in main body 2526. As shown in FIG. 25B, the proximal end of shank portion 2502 may be provided with a circumferential rib or barb 2530 for securing head portion 2506 to shank portion 2502 in a snap-fit manner. The head portion may be considered part of a rod coupling assembly. The head portion may be considered part of the implant, even though it is not directly engaging tissue.

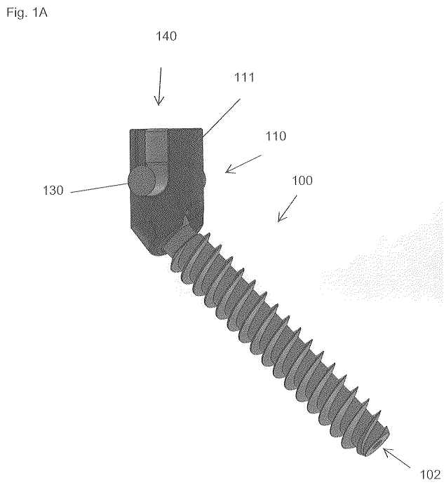

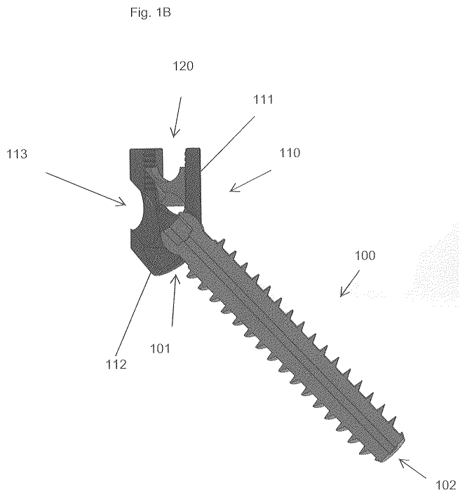

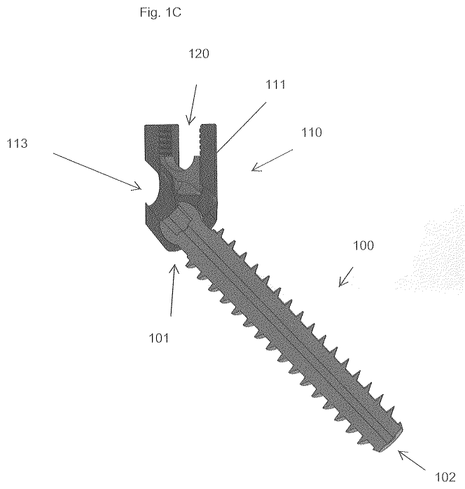

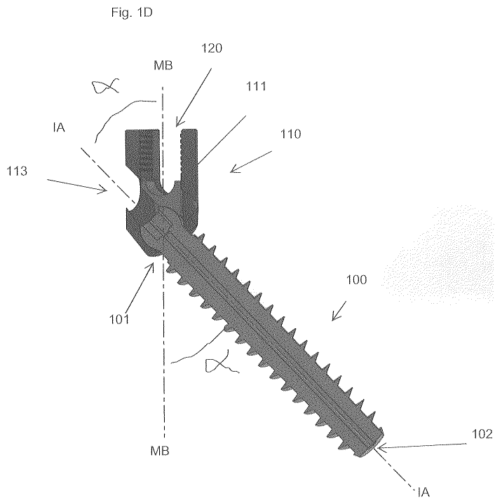

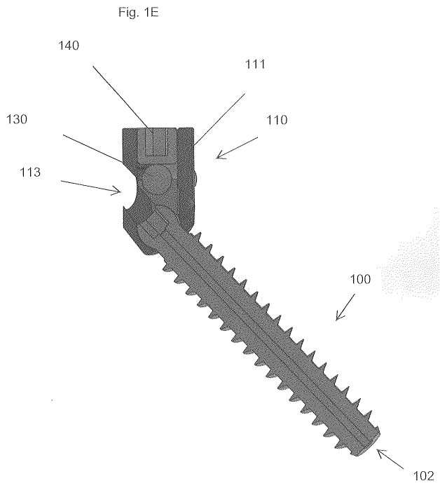

FIGS. 1A-1E herein illustrate an exemplary rod coupling assembly 110, which is shaped and sized to be secured to implant portion 100 as well as rod 130 (only a portion of which is shown). Assembly 110 includes main body 111, which may also be referred to herein as a tulip. Implant 100 has a rounded (optionally at least partially spherical) proximal end 101 that is shaped and sized to stably but movably interface with a curved inner surface 112 of main body 111 (shown in FIG. 1B) and a curved inner surface of implant stabilizing member 120 (e.g., a saddle), as shown in FIG. 1D. In the position in FIG. 1D (as well as the fully assembled views in FIGS. 1A and 1E), the main body 111 is rotatable to some degree relative to the proximal end 101 of implant 100 due to the spherical surfaces of the three components (implant 100, main body 111, and implant stabilizing member 120). The position of the curved surfaces is such that the implant axis “IA” (FIG. 1D) is not collinear with the main body axis “MB.” Preferably, the angle (as shown) is at least 15 degrees, and in some embodiments is between 35 and 55 degrees, optionally around 45 degree (e.g. between 42 and 48 degrees). The orientation of the spherical surfaces can thus impart and allow a favored or preferred angle between the main body (e.g., tulip) and the implant, while allowing for some rotational freedom between the two.

In the position shown in FIGS. 1A and 1C-1E, channel 113 in tulip 111 is in communication with and aligned with the internal channel in the implant, which includes distal port 102. This allows, if desired, an agent to be delivered through the tulip and through the implant and into the subject. The channel or port 113 in the tulip also allows access to the rotational drive mechanism such as a hex or torx drive.

Once implant stabilizing member 120 (e.g., saddle) is advanced to the position shown in FIG. 1D, rod 130 may be positioned relative to the rod channel in the tulip such that the rod is extending through the rod channel. FIGS. 1A and 1E illustrate a portion of a rod that is positioned in the tulip. Rods and tulips are at a high level known in the art. To secure the rod in place, rod stabilizing member 140 (e.g., a threaded set cap) may be advanced via threaded rotation into the main body until it engages the rod 130 and help secures it in place, as shown in FIGS. 1A and 1E. Implant stabilizing member 120 also has a proximal curved surface (as shown in FIG. 1B) configured to stably interface with a portion of the rod, as shown in the sectional view of FIG. 1E. In an exemplary use, rod 130 may be extending in a direction that is generally considered superior-to-inferior, and may be part of a spine stabilization system that includes additional bone anchors (“implant”). Once the rod is secured, the entire assembly, including the implant, is essentially a secure and stable construct.

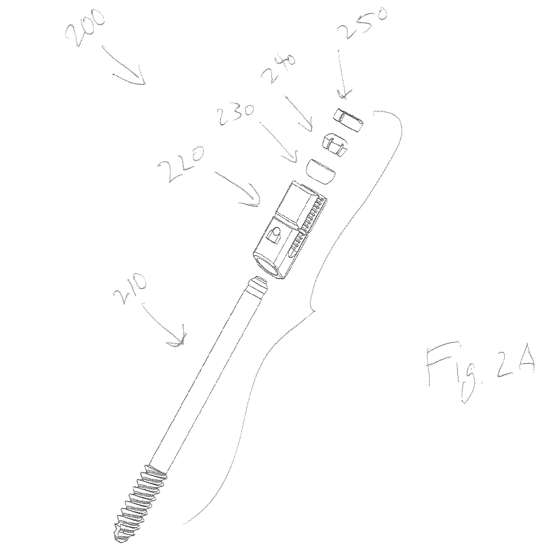

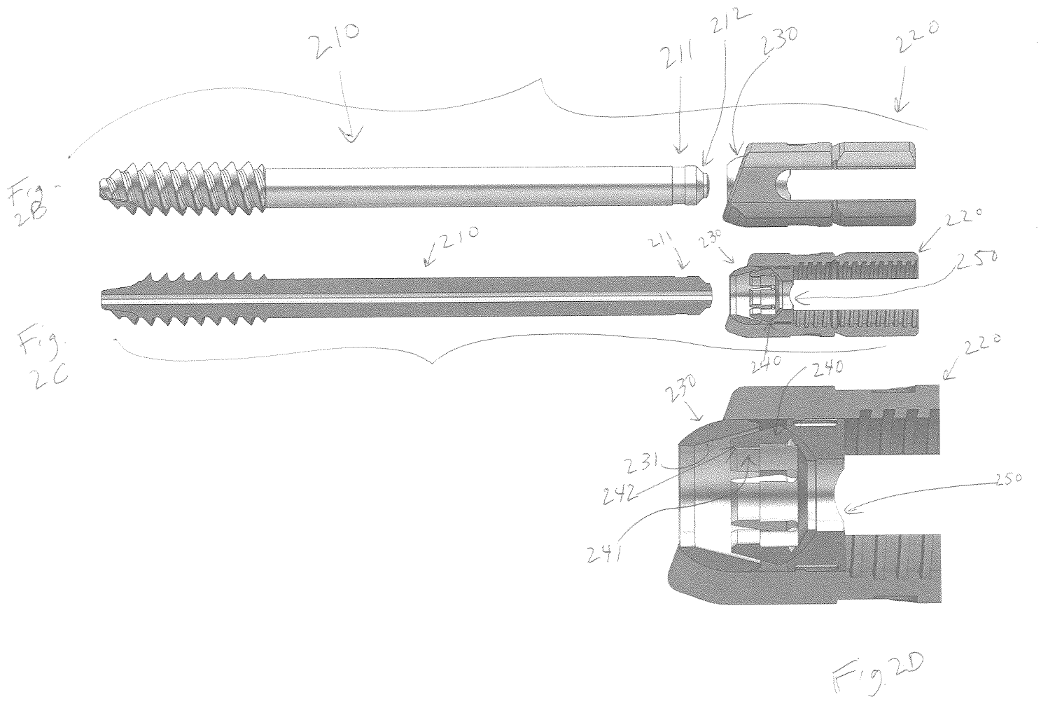

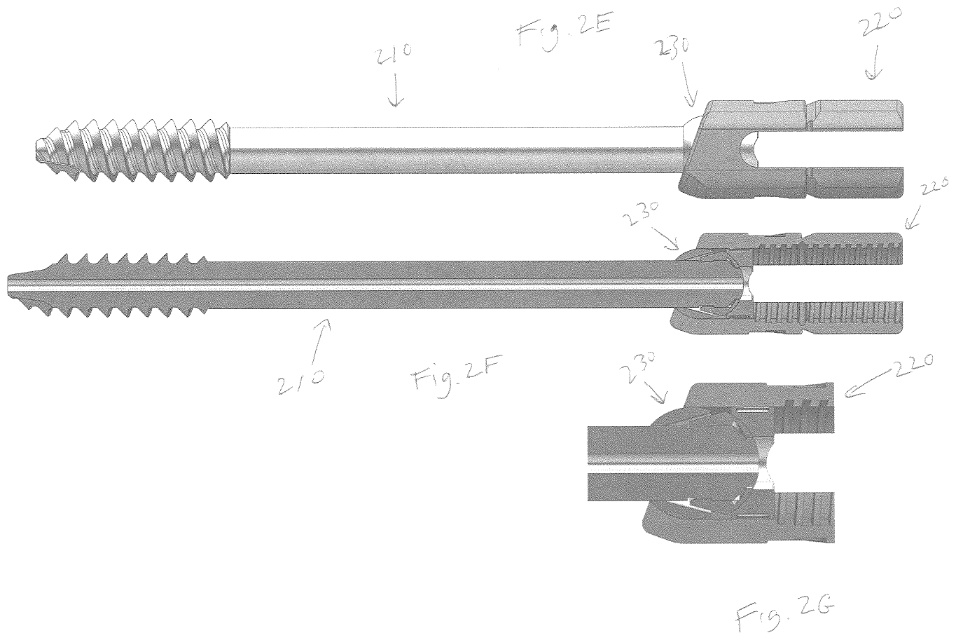

FIGS. 2A-2J illustrate a portion of an exemplary rod coupling assembly 200, but excludes illustrating the rod and set cap (e.g., a set cap 140 from FIGS. 1A-1E) for clarity. FIGS. 2A-2J illustrate the portion of the rod coupling assembly that illustrates how the main body or tulip 220 is secured relative to the implant portion 210. In any of the embodiments here, a rod can be secured relative to the tulip 220 using any of the disclosure herein, such as rod stabilizing member 140 shown in the embodiment in FIG. 1A. FIG. 2A shows an exploded view of rod coupling assembly 200, which may be considered to include a proximal region of implant portion 210. The rod coupling assembly 200 includes main body or tulip 220, distal collet portion 230, proximal collet portion 240, and saddle 250. Implant 210 may be at least partially threaded, as shown, and can be sized and shaped to anchor into bone.

The tulip 220 is configured to receive in a channel therein both spherical collet portions and the saddle, as shown in FIG. 2D. Proximal collet portion 240 includes slits formed therein, as shown. In FIGS. 2B, 2C and 2D, the split collet is disengaged from the mating tapered inner surface 231. The proximal portion of implant portion 210 may then be advanced within the collets, with the implant proximal end causing the collet fingers to deflect outward, or split. Continued advancement of the implant portion causes the collet fingers to deflect back inward about the implant groove 211, as is shown in FIGS. 2E-2G.

Saddle 250 may include one or more slots (shown in FIG. 2A) that are sized and configured to interface with swaged bosses to limit the amount of travel relative to the tulip.

The rod, not shown for clarity, may then be advanced through tulip 220, and a threaded set cap, not shown for clarity, can be advanced distally, causing the rod to be pushed against saddle 250, which compresses the collet, as shown in FIGS. 2H-2J. The implant proximal portion is now secured relative to the rod coupling assembly, and the rod is stably coupled to the rod coupling assembly.

In the embodiments in FIGS. 2-7 and 11 herein, once the desired angle is obtained between the tulip and the implant, the set screw is advanced, causing the set screw and the rod to advance the saddle against the collet. The slotted collet is then compressed against the tulip, which compresses and secures the collet against the proximal portion of the implant. This is the general method by which the assemblies are secured to the implant as well as the rod.

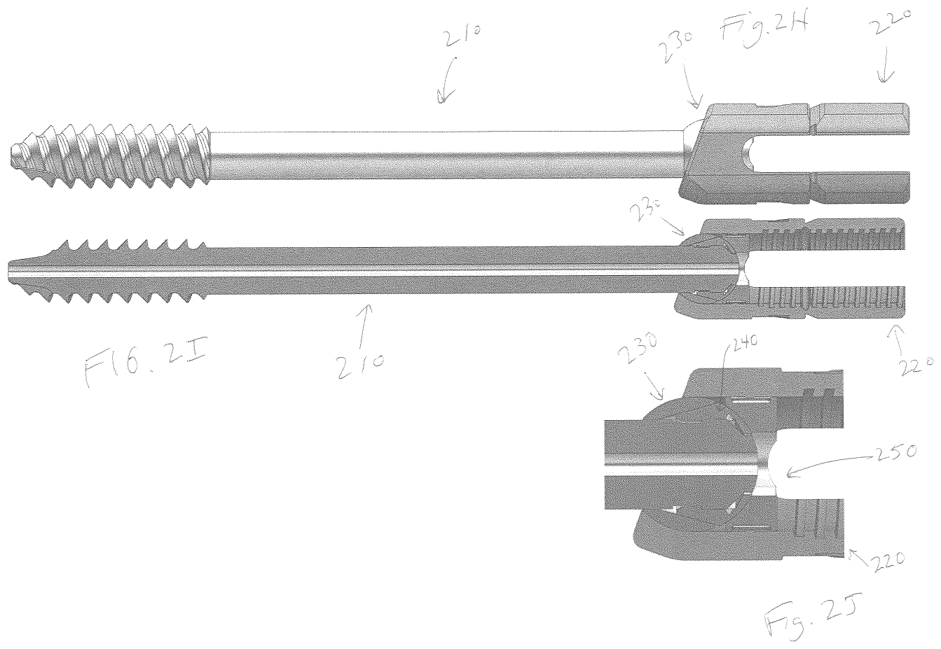

FIGS. 3A and 3B illustrate a variation on the embodiment in FIGS. 2A-2H (similar parts being similarly labeled), wherein the rod coupling assembly includes a saddle and proximal collet that are threaded to interface with threaded tool 260. Once the threaded tool 260 is threaded into the saddle and collet, as shown in FIG. 3A, the tool is then pulled proximally with respect to the tulip 220′, pulling the saddle and collet proximally and disengaging from the distal spherical collet. This allows the saddle to be retracted proximally, which provides the option to remove the tulip if needed (e.g., if the implant portion were to become loose). The tulip can also have a side channel (as in FIGS. 1A-1E) that can provide access to the proximal end of the implant if needed, such as if the tulip is rotated and the physician cannot get access to the tulip main channel. The distal collet portion 230 (or 230′) allows for favored angle rotation of the tulip 220 relative to the implant portion 210.

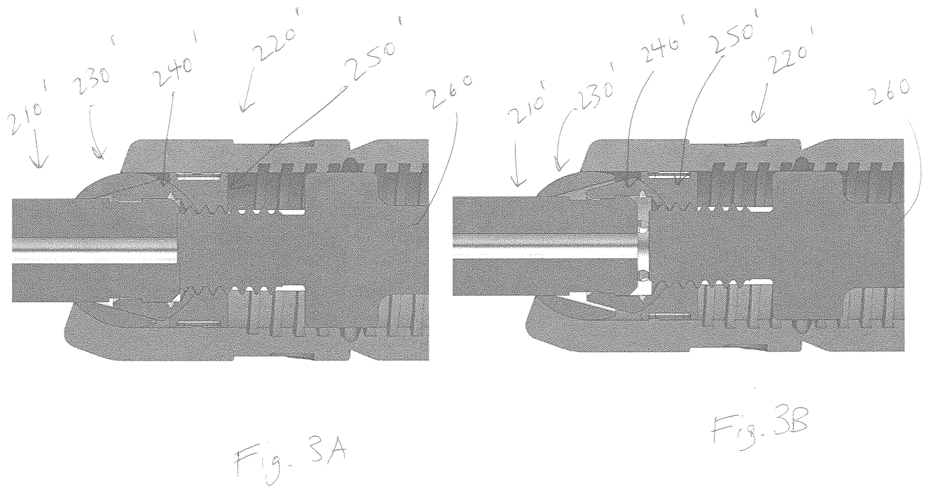

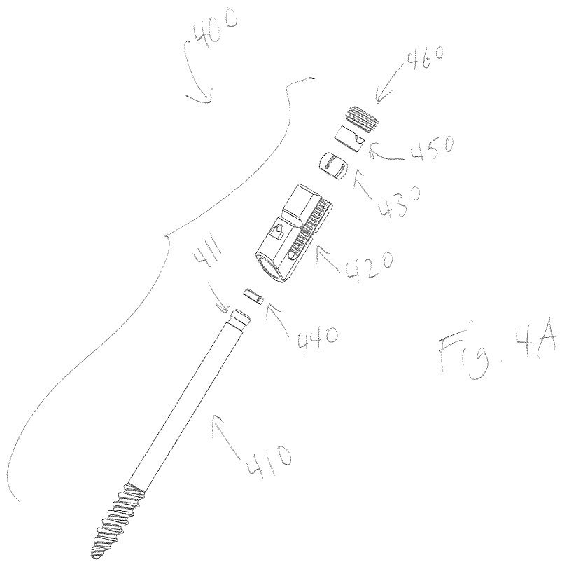

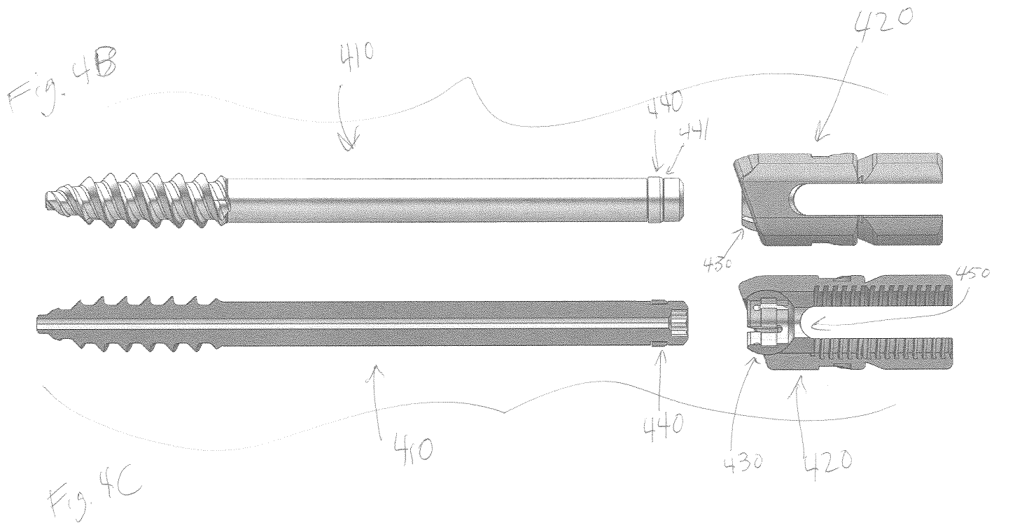

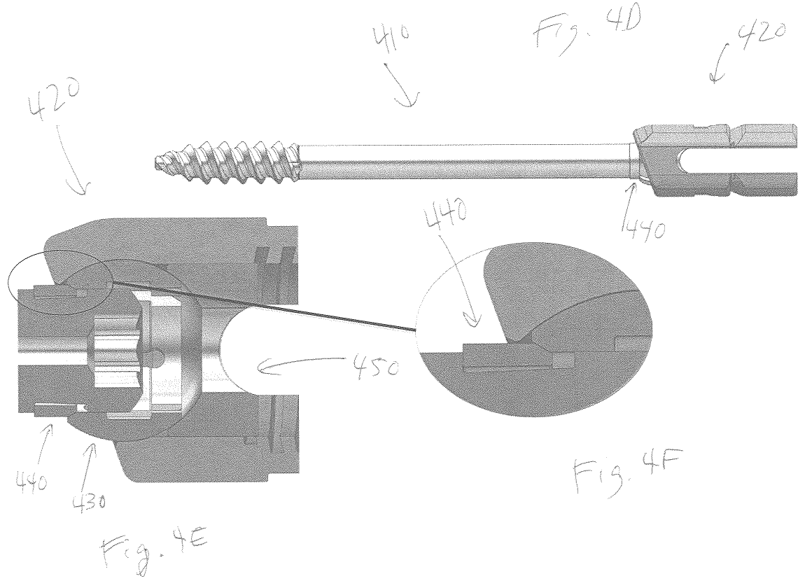

FIGS. 4A-4H is an exemplary embodiment of a rod coupling assembly 400 that includes main body or tulip 420, spherical collet 430 (shown with slits in FIG. 4A), saddle 450, set cap 460, and split annular member 440. Implant portion 410 is also shown, a proximal region of which includes groove 411 or other similar type of depression or recessed region formed therein. The implant 410 may come assembled with split annular member 440 (e.g. split ring), such as in the configuration in FIGS. 4A and 4B. Main body or tulip 420 houses spherical member 430 and saddle 450. A rod that can pass through the tulip is not shown for clarity, but threaded set cap 460 can be advanced within the main body and advanced until it secures the rod in place, details of which are described elsewhere herein.

Split annular member 440 has a tapered proximal surface 441, shown in FIGS. 4B and 4C. When the implant portion 410 and annular member 440 are advanced into the tulip 420 and spherical collet 430, as shown in FIGS. 4D, 4E and 4F, split annular member 440 is pushed distally into the deepest part of the tapered implant groove 411. When set-cap 460 is torqued, split annular member 440 is pushed into the shallow portion of the tapered implant groove 411 and into the groove within the spherical collet 430, as shown in FIGS. 4G and 4H.

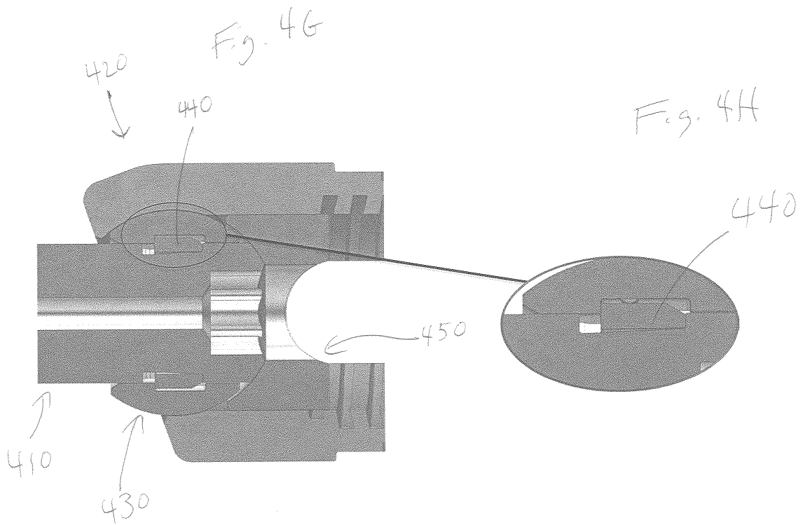

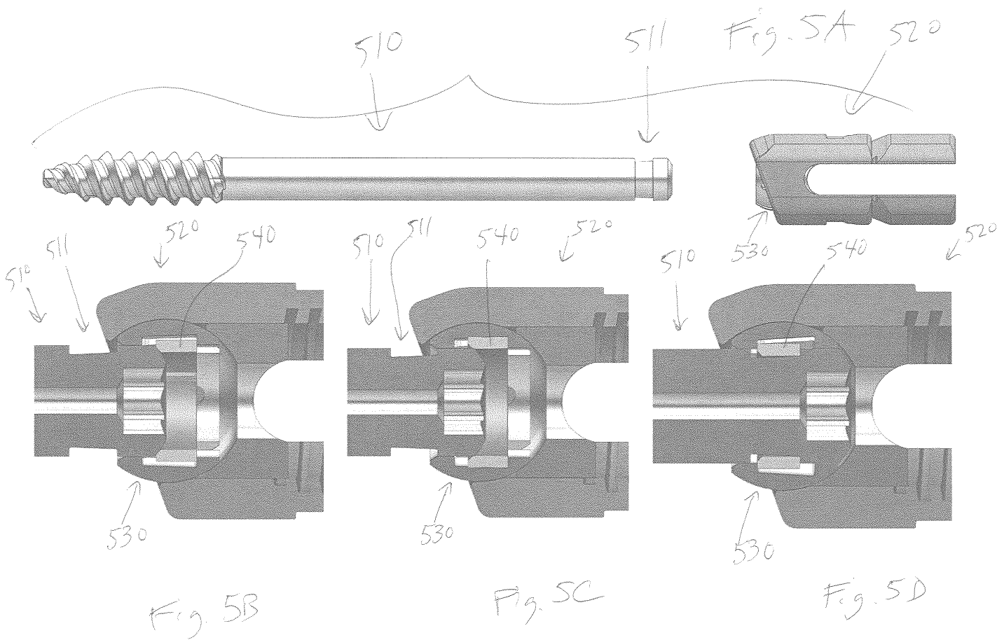

FIGS. 5A-5D illustrate an exemplary rod coupling assembly that is similar to the embodiment in FIGS. 4A-4H, and similar or the same parts are similarly labeled. All disclosure from the description of FIGS. 4A-4H can be expressly incorporated into the embodiment of FIGS. 5A-5D unless indicated to the contrary. In FIGS. 5A-5D, split annular member 540 is relatively larger than in FIGS. 4A-4H, and can be pre-assembled within a groove in spherical member 530, as shown in FIGS. 5B-5D. The movement of the annular member in this embodiment is opposite that in FIGS. 4A-4H. As the proximal region of implant 510 is advanced into the spherical member 530, annular member 540 is pushed proximally into the deepest part of spherical member groove, as shown in FIG. 5C. As the proximal region of implant 510 is further advanced, annular member 540 snaps into implant groove 511. As the set cap is torqued, split annular member 540 is pushed into the shallow portion of the spherical member groove, as shown in FIG. 5D.

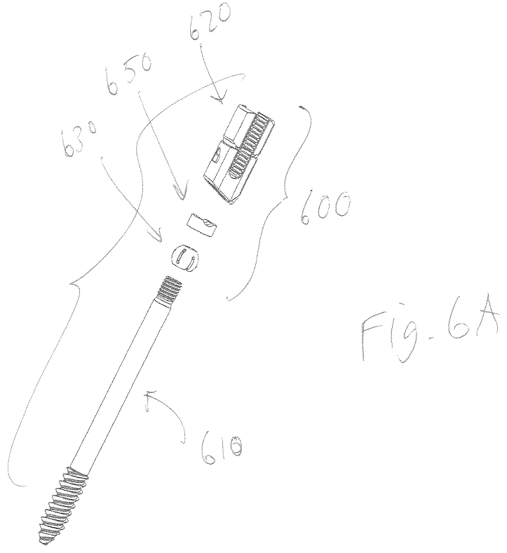

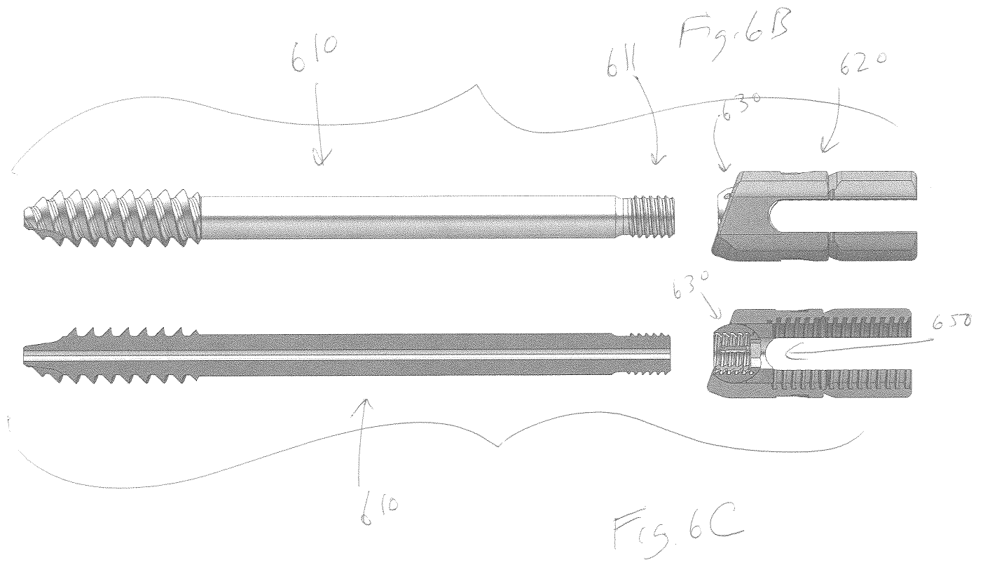

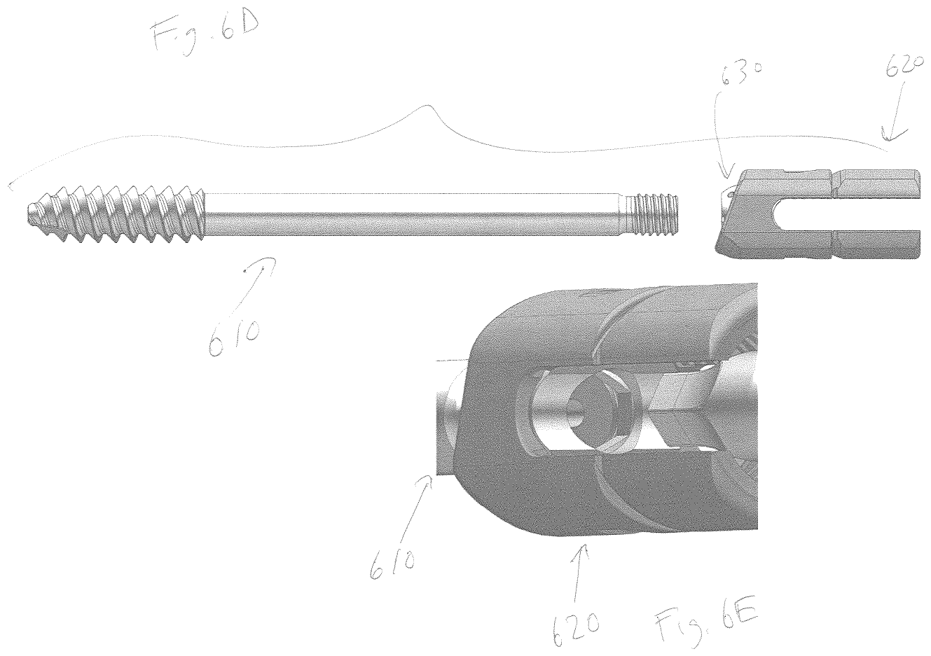

FIG. 6A-6E illustrate an embodiment of a rod coupling assembly 600 that includes main body or tulip 620, saddle 650, spherical collet 630, and a set cap (not shown). Threads 611 on the proximal region of implant 610 (FIG. 1B) interface with internal threads in spherical collet, which are shown in FIG. 6C. The main body or tulip 620 and collet 630 assembly is threaded onto the implant, and the tulip 620 driver engages hex or torx in the spherical collet, as shown in FIG. 6E. The thread can be a saw tooth thread. The spherical collet has slits formed therein so that as the spherical collet is rotated it is slightly compressed radially. A rod can then be positioned through the tulip, and a set cap can be torqued to secure the rod in place.

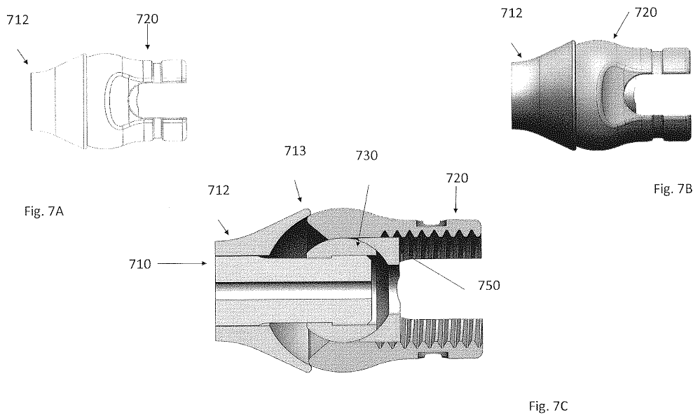

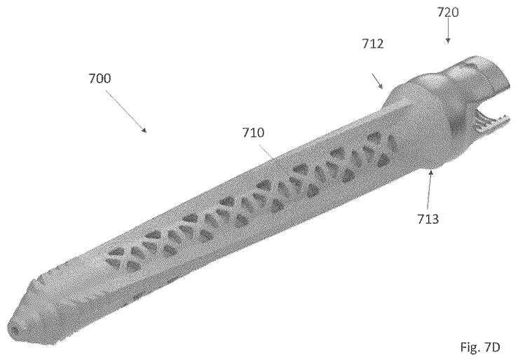

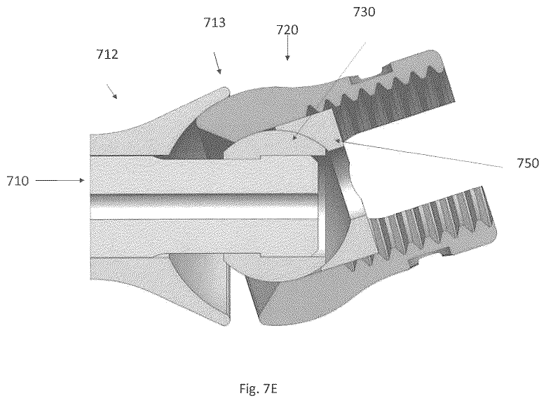

When a rod is secured to a rod coupling assembly of an anchoring implant (such as any of those described herein), bending loads may be applied to the tulip and to the implant. It may be beneficial for the rod coupling assembly and/or implant to be configured and/or sized to help resist the bending loads. FIGS. 7A-7E illustrate a merely exemplary rod coupling assembly in which a portion of the implant, or implant portion, interfaces with the main body, or tulip, and in particular, provides support for the tulip to provide more resistance to bending loads. In FIG. 7C, shank 710 of the implant 700 is an example of an elongate member of the implantable portion, such as any of the elongate inner members (e.g., shanks) in PCT published application WO2020/168269, the disclosure of which is incorporated by reference herein. Outer sleeve 712 refers generally to an outer member that is disposed at least partially about the inner member or shank 710. FIG. 7D illustrates a non-limiting example of an implant 700 that includes an outer sleeve 712 and an inner elongate body 710 (e.g., shank portion). The sleeve includes a proximal region 713 that has a configuration that extends radially outward and extends such that its proximal end is disposed radially outside of and about at least a portion of the tulip 720 and provides support to the tulip. The tulip may also act a locking mechanism for the outer sleeve.

The sleeve 710 in this exemplary embodiment may be any of the outer elongate members herein, including PCT published application WO2020/168269, such as body 2504 in FIG. 25B. Additionally, any of the outer elongate members (e.g., body 2504 in FIG. 25B in PCT published application WO2020/168269), may include a proximal region that extends radially outward, as does the sleeve 710 in FIGS. 7A-7E. The implants in this embodiment may take a wide variety of shapes and configuration and may include a proximally extending region 713 that is sized and conjured to interface with an outer surface of the tulip.

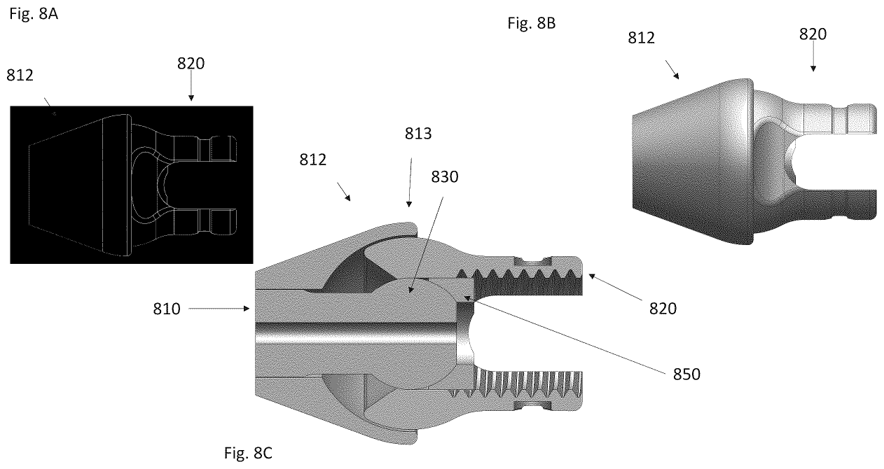

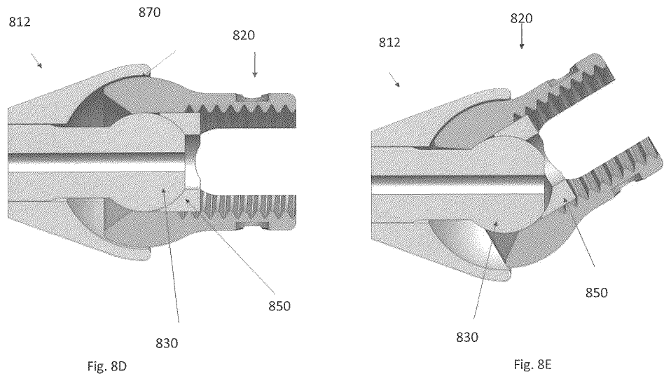

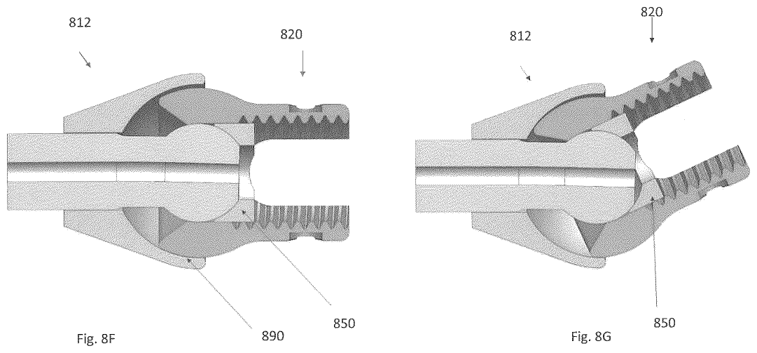

FIGS. 8A-8G illustrate an embodiment of a rod coupling assembly that is similar in some ways to the embodiment shown in FIGS. 7A-7E, and may incorporate any suitable disclosure by reference into the embodiment. In this embodiment, a sleeve portion 812 of the implant extends around tulip 820 to a greater extent than in FIGS. 7A-7E, providing more support and more resistance to bending loads. The spherical component in this embodiment is part of implant as implant spherical element 830, shown in FIG. 8C. FIGS. 8D and 8E illustrate clearance 870 between tulip 820 and implant sleeve 812 under normal or no loading. FIGS. 8F and 8G illustrates an extreme load scenario, and illustrates how outer sleeve 812 acts as a brace and prevents excessive deflection of the neck region of the inner shank 810. The rod and set screw are not shown for clarity.

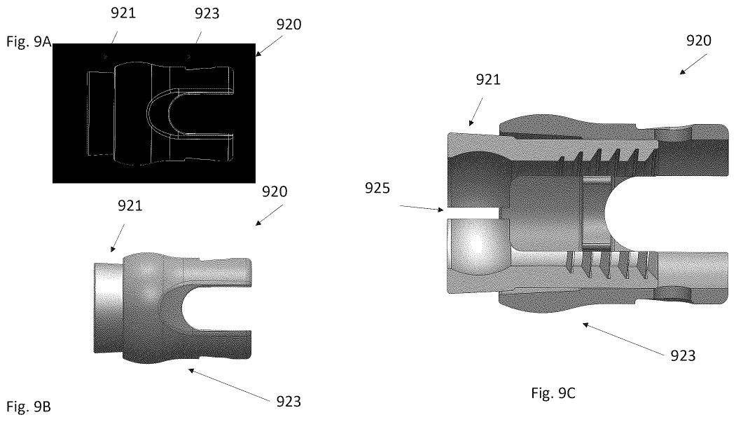

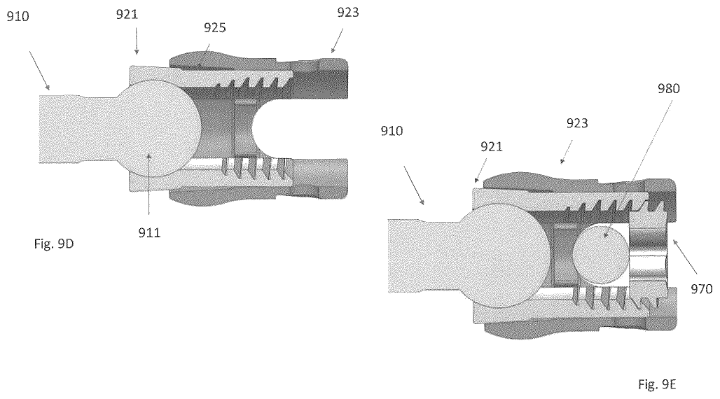

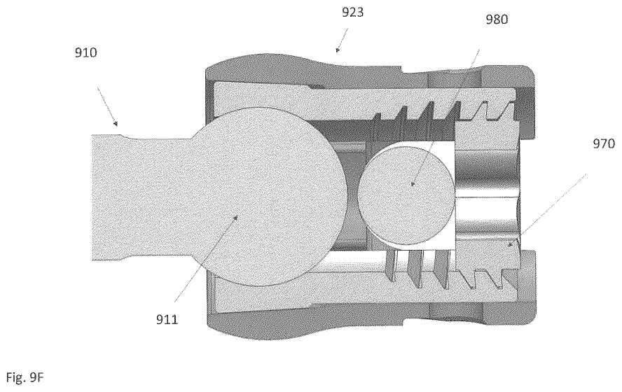

As discussed herein, some rod coupling assemblies may be under bending loads once implanted. FIGS. 9A-9F illustrate an exemplary tulip that includes an inner tulip member and an outer tulip member. Tulip 920 shown in FIGS. 9A-9F may be used with implants other than those shown or described herein. Tulip 920 includes outer tulip 923 and inner tulip 921. The tulips in FIGS. 9A-9F may be incorporated into any suitable embodiment herein unless indicated to the contrary. FIGS. 9A-9C illustrate the tulip member 921 and 923 without the rest of the rod coupling assemblies. FIG. 9D illustrates an exemplary implant portion 910 with a spherical member 911 being positioned within inner tulip 921. As shown, clearance 925 between outer tulip 923 and inner tulip 921 in the unlocked position allows the spherical member 911 to snap into the inner tulip interface, as shown. Then, in FIG. 9E, after rod 980 placement, set cap 970 tightening causes inner tulip 921 to be pulled and moved proximally relative to outer tulip 923, as shown. As shown in FIG. 9F, the taper interface between outer tulip 923 and inner tulip 921 allows the tulip assembly to lock onto the spherical member 911. In this final configuration, the outer tulip helps prevent inner tulip splaying, to which some tulips may be susceptible under bending loads. As shown in the figure, the rod coupling assembly may also include a saddle that can be advanced to the spherical member, such as any of the saddles herein. This embodiment of an example of an inner tulip member that is configured to be moved axially relative to an outer tulip member when in use.

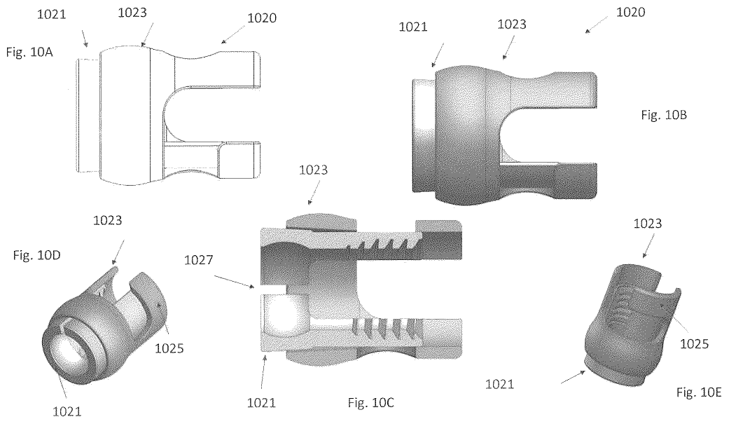

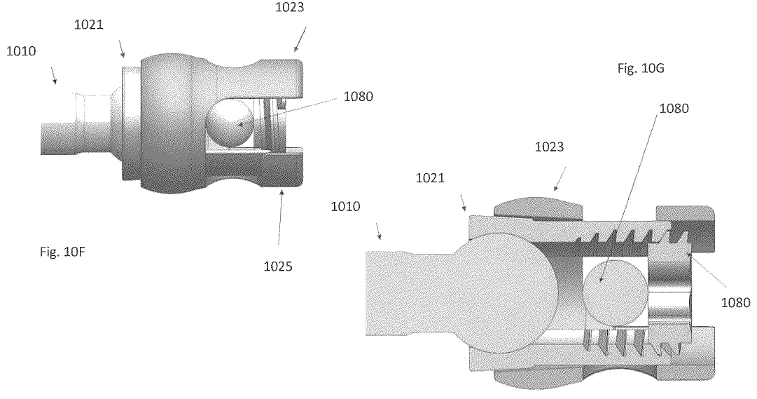

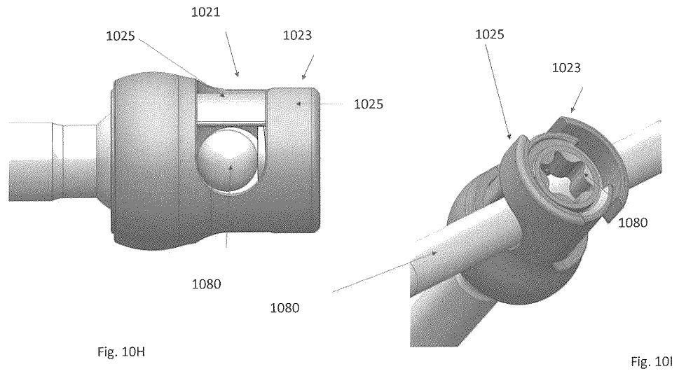

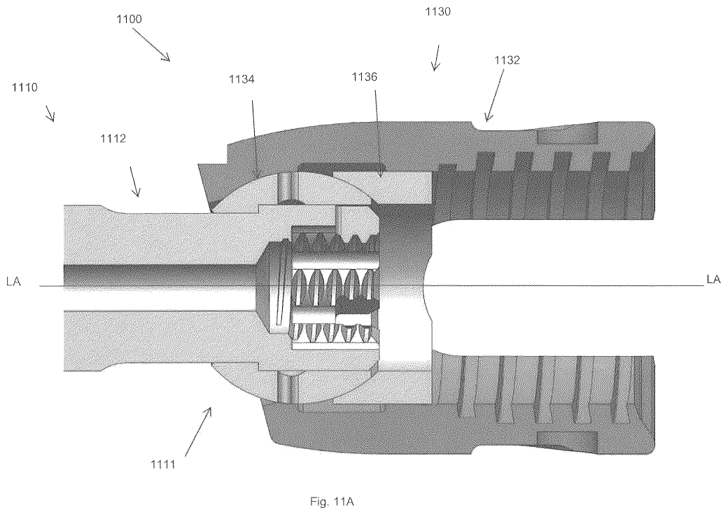

FIGS. 10A-10I illustrate an exemplary embodiment of a rod coupling assembly that is similar to the embodiment shown in FIGS. 9A-9F with inner and outer tulip members. Any of the suitable disclosure from the embodiment in FIGS. 9A-9F may be incorporated into the disclosure of 10A-10I unless indicated to the contrary, such as the set cap threading causing the inner tulip to be drawn proximally within the outer tulip. As shown, and as is the case in the embodiment in FIGS. 9A-9F, inner tulip 1021 has a distal end with at least one slit 1025 therein, allowing it to be expanded and collapsed to some extent during axial movement. One difference in this embodiment is the configuration of the outer tulip 1023, and the manner in which it may be rotated (for example only, a quarter or ¼ turn) prior to final set cap 1080 tightening. Rotating outer tulip 1023 causes a portion 1025 of outer tulip 1023 to extend over a region of inner tulip, as shown, which can provide more radial support to the tulips arms, and can reduce the likelihood of inner tulip splaying. In other embodiments, the outer tulip and or the inner tulip could have different configurations, and could be rotated more or less than a quarter turn (90 degrees), such as between 0 and 180 degrees, such as from 5 to 135 degree, such as from 45 to 135 degrees, such as 90 degrees.

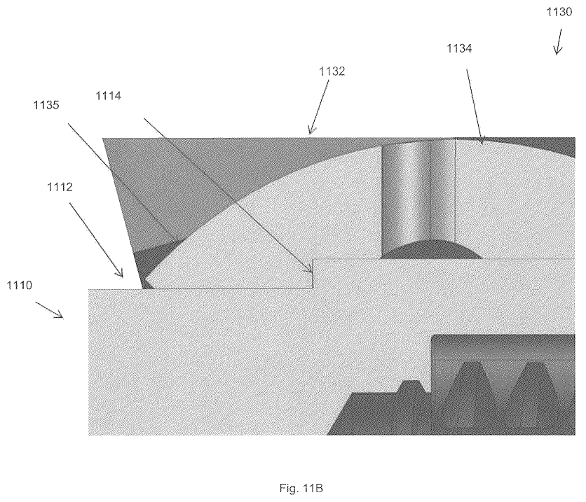

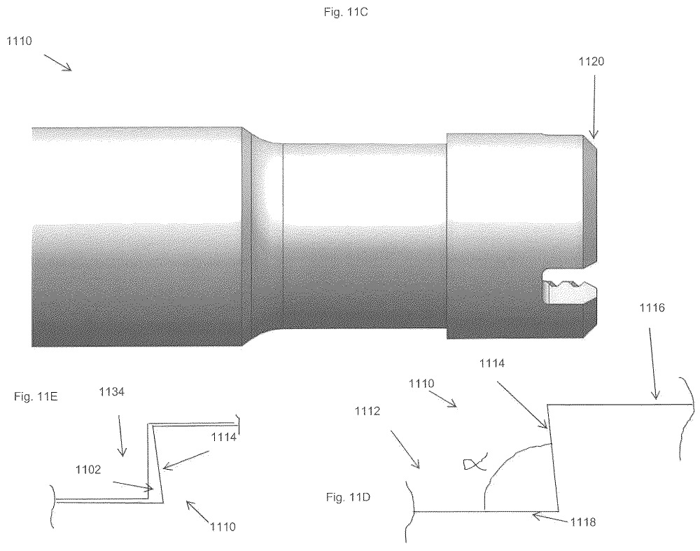

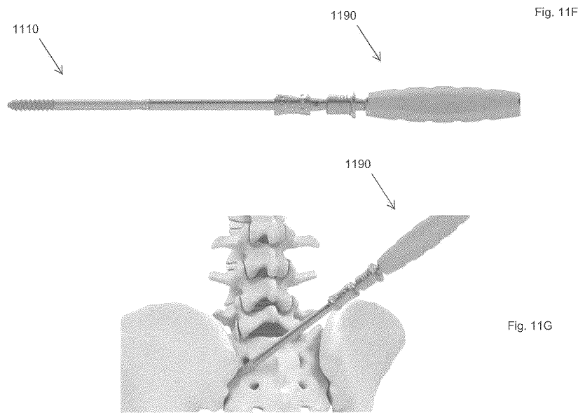

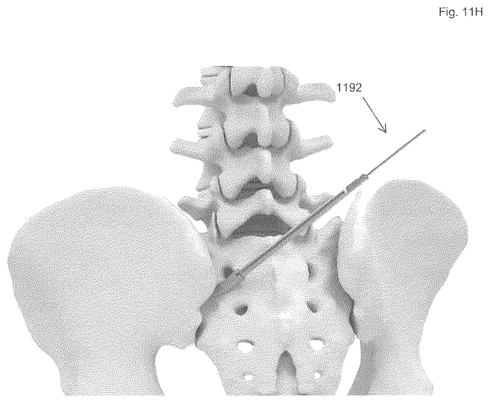

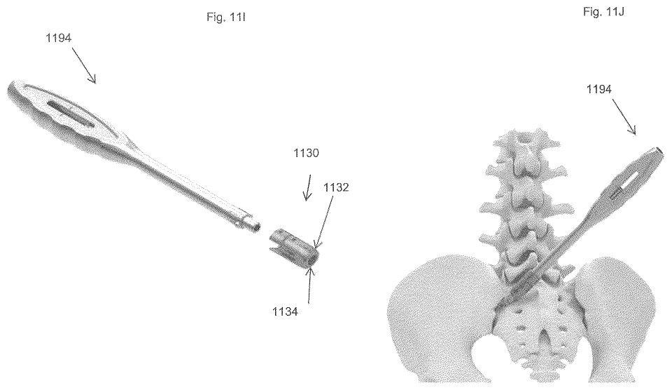

FIGS. 11A-11J illustrate an additional exemplary embodiment of rod coupling system or assembly 1100 that includes exemplary bone implant 1110 and tulip assembly 1130. It is understood that alternative systems may include bone implant 1110 and other types of tulip assemblies (including those not described herein). It is also understood that alternative systems may include tulip subassembly 1130 and other types of bone implant (including those not described herein). It is further understood that alternative systems may include one or more features of bone implant 1110 and other types and features of tulip subassemblies (including those not described herein). It is also understood that alternative systems may include one or more features of tulip subassembly 1130 and other types and features of bone implants (including those not described herein).

System 1100 and other similar systems herein that include a tulip assembly and bone implant may also be referred to herein as a rod coupling assembly or system, as the system may be used to couple or secure a stabilizing rod relative to the tulip assembly and bone implant.

System 1100, bone implant 1110 and tulip assembly 1130 may be understood to include other features set forth herein even if the textual description thereof does not expressly disclose those features.

An exemplary aspect of this embodiment is that an interface between a collet and a proximal region of the bone implant is sized and configured so as to reduce the likelihood of the distal end of the collet from expanding and possibly becoming loose in response to axial forces (e.g., proximal forces) on the tulip. For example, proximal forces on the tulip from rod movement may translate to the collet, which may tend to undesirably cause the collet distal end to loosen or expand over time.

Additionally, the collect and implant interface in the exemplary embodiment in FIGS. 11A-11J is configured to resist or minimize the likelihood of becoming loose from axial forces caused by rod reduction. Rod reduction can be generally described as when a physician may position the rod proud of the tulip and reduces it (i.e., forces the rod into the implant tulip) in order to get a correction in anatomy. This step occurs before the collet is tightened in place. These reductions exert a great amount of force on the implant, and because the collet is not yet tightened, the collet may come apart. The collet and implant interface in FIGS. 11A-11J is an example of such an interface that is sized and configured to ensure, or increase the likelihood of, the collet remaining closed during these reductions.

To reduce the likelihood of collet loosening or expansion in response to these forces and in these scenarios, the implant undercut creates or produces a gap 1102 (FIG. 11E) between the non-parallel surface 1114 and the collet surface that faces surface 1114. The collet surface may be orthogonal to the long axis. When an axial force is applied to tulip, gap 1102 provides space so that less distally directed force is applied to the vertical surface of collet, reducing the likelihood of collet expansion and loosening. The reduction in likelihood is compared to a system that may be alike in all ways but instead having a radially extending surface 1114 that is orthogonal to the bone implant longitudinal axis LA, or compared to systems in which a gap does not exist.

The undercut in the proximal end of the bone implant recess 1112 may be described in a variety of ways. The undercut may be described as a recessed collet receiving region 1112 in the proximal region of the implant that includes a radially extending surface (e.g., 1114) that is disposed at an angle less than ninety degrees relative to a bone implant longitudinal axis, the angle measured distally relative to the radially extending surface, as shown as angle alpha in FIG. 11D. In FIG. 11D, the angle is also shown relative to axially extending surface 1118, but axially extending surface 1118 need not be flat as is shown, but instead may be curvilinear. In any embodiments herein, however, surface 1114 or an extension thereof may form an angle with a long axis of the implant or an axis that is parallel with the long axis, which may be the same angle alpha in both cases.

The undercuts herein may also be described and claimed functionally herein, such as the radially extending surface angled so as to reduce the likelihood of a distal end of the collet expanding and becoming loose in response to an axial force (e.g., solely proximally directed), compared to a system alike in all ways but instead having a radially extending surface that is orthogonal to the bone implant longitudinal axis.

The undercuts herein may be described as including a proximal region that includes a radially recessed collet receiving region or area including a radially extending surface that is disposed at an angle less than ninety degrees relative to the long axis. The undercuts herein may be described as including a proximal region that includes a radially recessed collet receiving area that includes a distal axially extending surface (e.g., surface 1118) and a proximal axially extending surface (e.g., 1116), the distal and proximal axially extending surfaces on either side of a radially extending surface (e.g., 1114), the proximally axially extending surface disposed further radially outward from the long axis than the distal axially extending surface, an example of which is shown in FIGS. 11D and 11E.

The undercut may be considered as including radially extending surface 1114 and one or more surfaces extending therefrom, such as axially extending surface 1118 and axially extending surface 1116. In this example, the undercut includes a distal end of axially extending surface 1116 extending further distally than a proximal end of radially extending surface 1114, as is shown in FIGS. 11D and 11E.

As described herein, an axially extending surface is not necessarily parallel with a long axis of the implant, but rather may be at an angle thereto and still considered to be axially extending, or may even be curvilinear and still be considered to be axially extending.

Collet 1134 or any collet herein may include common collet features, such as linear slits or slots formed therethrough that allow for expansion and collapse of a distal end of the collet. This may allow the collet to be advanced over the proximal end of the implant, causing collet expansion. In some embodiments, the proximal end of the implant may facilitate collet expansion, such as by having chamfered surface 1120 (FIG. 11C), which can help expand the distal end of the collet as it is slid distally over the proximal end of the implant. Once the collet implant interface region 1135 (FIG. 11B) is advanced distally to the recessed region 1112, the collet implant interface region 1135 collapses into the recess 1112 due to the collapsibility of the collet distal end due to the slits/slots. Snapping or collapsing into place into the recessed region 1112 secures the collet in place relative to the proximal region 1111 of the implant, as is shown in FIGS. 11A, 11B, and 11E. Tulip 1132 may still be moved relative to the collet 1134 until a set screw is threaded into the tulip (internal threads shown in FIG. 11A) to secure the rod in place (not shown in FIGS. 11A-11I), but any rod herein may be positioned through the tulip side openings.

Collet implant interface region 1135 shown in FIG. 11B is an example of a radially inward protruding implant interface region of the collet, and is shown disposed and interfaced with recessed region 1112 of collet 1134. The radially extending surface of the collet 1134 that is shown in FIG. 11E that is facing implant surface 1114 may be orthogonal to a central axis of the collet. In other embodiments it may be disposed at an angle relative to a long axis.

In some embodiments, the radially extending surface of the implant interface region of the collet (e.g., as shown in FIG. 11E) is orthogonal to a longitudinal axis of the collet.

In some embodiments, the radially extending surface of the recessed collet receiving region of the implant is disposed at an angle (e.g., angle alpha in FIG. 11D) that is less than 85 degrees relative to the longitudinal axis of the implant. In some embodiments, the angle alpha may be greater than 45 degrees relative to the longitudinal axis of the implant. In some embodiments, the angle alpha is greater than 75 degrees relative to the longitudinal axis of the implant. In some embodiments, the angle is from seventy degrees to eighty-nine degrees relative to the longitudinal axis of the implant.

In some embodiments, the recessed collet receiving region (such as region 1112) has a depth from 0.1 mm to 2.0 mm relative to an outer dimension of the implant axially adjacent to the recessed region 1112.

In any of the embodiments herein the bone implant proximal region may be void of an external thread, such as is shown in the example in FIGS. 11A-11J.

In some embodiments, the recessed collet receiving region of the implant has an annular configuration about the bone implant, such as is shown in FIG. 11C.

Even if not shown herein, a collet of any of the systems herein may have a partially spherical configuration, and the tulip may have a corresponding inner surface. The collet and tulips surfaces may be together sized and configured to interface with each other when the collet is disposed within the tulip. The tulip inner surface and the collet outer surface may be both sized and configured to cause the tulip to be movable relative to the collet in a movable state, such as in the embodiment in FIGS. 11A-11J.

Any of the tulip assemblies or rod coupling assemblies herein may include further a saddle sized and configured to be positioned within the tulip and at least partially proximal to the collet. The saddles herein may have a distal region with an inner curved surface shaped to mate with a curved proximal region of the collect. The saddles herein may have a proximal end with a recessed region having a configuration shaped to interface with an outer surface of the elongate rod (e.g., as shown in the embodiment in FIGS. 11A-11J).

Any of the collets herein may have one or more openings formed therein. Threaded a set screw into the tulip in FIG. 11A applies a force to a rod (not shown) and thereby to the saddle. This, along with the collet/tulip interface, causes compression of the collet around the implant proximal end. Once the tulip is at the desired angle, the set screw can be advanced as set forth herein and essentially stabilize the tulip relative to the rod and to the implant.

While exemplary bone implants are shown herein, bone implants that may be part of the rod coupling assemblies herein may be configured and sized as a wide variety of bone implants. For example, bone implants herein may be threaded along at least a portion of their lengths, or they may be non-threaded. The bone implants herein may be configured to be advanced into one or more of an ilium, or a sacrum or other vertebrae.

One aspect of this disclosure is a bone implant, with or without a tulip assembly associated therewith. The bone implant may have an elongate body with a long axis and a proximal region, wherein the proximal region is sized and configured to be coupled within a collet of a tulip assembly. The proximal region of the implant may include a radially recessed collet receiving region (e.g., region 1112) including a radially extending surface (e.g., surface 1114) that is disposed at an angle less than ninety degrees relative to the implant long (or central) axis. The collet receiving region of the implant may also include a distal axially extending surface and a proximal axially extending surface, the distal and proximal axially extending surfaces on either side of the radially extending surface, wherein the proximally axially extending surface is disposed further radially outward from the long or central axis than the distal axially extending surface.

The proximal ends of any bone implants herein may have a chamfered configuration, such as is labeled as 1120 in the example shown in FIG. 11C.

A distally axially extending surface (e.g., surface 1118) may be at an angle from zero to twenty-five degrees, inclusive, relative to the implant long axis.

Any of the bone implants herein may include a proximal region that has an inner lumen with an inner thread, such as is shown in FIG. 11A. In some embodiments that are coupled as modular tulip assemblies, the inner thread may be sized and configured to interface with an outer thread on a guide rod that may facilitate advancement of a modular tulip assembly including a collet over the proximal region of the elongate body.

Any of the undercut ledge configurations herein may also be described similarly to a cliff with an upper surface, at least a portion of which extends distally relative to a radially extending wall portion of the cliff.

Tulips assemblies herein may be coupled to the implant prior to insertion, or they may be modular and coupled to the implant after the implant is at least partially implanted into bone.

Whether modular of coupled to the implant prior to bone implant implantation, the tulip assemblies may include any of the features of any tulip assemblies herein.

The tulips herein may include any suitable features common to existing tulips. FIGS. 11A-11J illustrate an exemplary tulip with first and second side openings that are sized and configured to receive an elongate rod therethrough. FIGS. 11A-11J illustrate an exemplary tulip assembly with a collet that includes a radially inward protruding implant interface region (e.g., region 1135), the protruding implant interface region including a radially extending surface disposed at an angle relative to a collet longitudinal axis and an axially extending surface that meets the radially extending surface, as shown. The radially inward protrusion facilitates the collapse of the collet distal end towards the recessed region of the bone implant proximal region as the collet is advanced over the proximal region of the bone implant (or the implant is moved axially within the collet). Any of the tulip assemblies herein may include a saddle that is sized to be disposed within the tulip and at least partially proximal to the collet. Any saddle may have a distal region with an inner curved surface shaped to mate with a curved proximal region of the collect. Any saddles herein may have a proximal end with a recessed region with a configuration shaped to interface with the elongate rod.

Any of the tulip assemblies herein may include a collet having one or more openings or slits therein that allow and cause the collet to apply a radially inward clamping force in response to forces applied to the collet from the tulip and the saddle.

Any of the tulip assemblies herein may include a radially inward protrusion (e.g., implant interface region 1135) that has a height from 0.1 mm to 2.0 mm, the dimension of which may be considered to be substantially the same as a depth as any of the recessed implant regions herein.

Any of the collets herein may include a protrusion that includes a radially extending surface that is orthogonal to a collet long axis, such as is shown in exemplary FIG. 11E.

Additional disclosure of system components and features disclosed in U.S. Pat. Nos. 8,84,5693 and 9,655,656 may be relevant to disclosure herein, and the entire disclosures thereof are incorporated by reference herein for all purposes.

FIGS. 11F-11J illustrate merely exemplary method steps and delivery tools for coupling a modular tulip assembly to an implant.

FIGS. 11F and 11G illustrate implant 1110 being implanted using implant delivery tool 1190. After removing implant delivery tool 1190, guide rod 1192 is coupled to the proximal region of the bone implant such that the guide rod extends proximally from the bone implant, as shown in FIG. 11H. After removing the guide rod, a tulip delivery tool 1194 that is coupled to a tulip assembly 1130 is used to advance the tulip assembly over the guide rod, as shown in FIGS. 11I and 11J. The method may further include advance the collet against a proximal end of the bone implant proximal region and causing a distal end of the collet to expand as it is advanced over the bone implant proximal region. The method may further include continuing to advance the collet over the bone implant proximal region until a radially inward protrusion of the collect moves radially inward into a recessed region of the bone implant, the recessed region of the bone implant having a proximal end with an undercut ledge configuration.

In some embodiments the bone implant is implanted with the tulip assembly already coupled thereto.

1. (canceled)

2. A method of securing a bone implant to a rod, the method comprising:
advancing a bone implant into bone with a bone implant delivery tool that is coupled to a proximal region of the bone implant;
disengaging the bone implant delivery tool from the bone implant and removing the bone implant delivery tool;
coupling a guide rod to the proximal region of the bone implant such that the guide rod extends proximally from the bone implant;
coupling a tulip assembly delivery tool to a tulip assembly, the tulip assembly including a tulip, a collet at least partially disposed within the tulip, and a saddle within the tulip and at least partially proximal to the collet;
using the tulip assembly delivery tool to advance the tulip assembly over the guide rod;
advancing the collet against a proximal end of the bone implant proximal region and causing a distal end of the collet to expand as it is advanced over the bone implant proximal region;
continuing to advance the collet over the bone implant proximal region until a radially inward protrusion of the collect moves radially inward into a recessed region of the bone implant, the recessed region of the bone implant having a proximal end with an undercut ledge configuration.

3. The method of claim 2, further comprising positioning a rod through a side opening in the tulip, and screwing a set screw into the tulip until the set screw applies a stabilizing force on the rod and until the tulip is secured in place relative to the bone implant.

4. The method of claim 2, wherein the tulip assembly is modular and is adapted to be delivered separately from the bone implant and movably secured to the bone implant after the bone implant has been at least partially implanted within bone.

5. The method of claim 2, wherein the step of coupling a guide rod to the proximal region of the bone implant further comprises engaging an internal thread of the implant proximal region with the guide rod.

6. The method of claim 2, wherein the step of advancing the collet against a proximal end of a proximal region of a bone implant further comprises positioning a radially inward protruding implant interface region of the collet within a radially recessed collet receiving region of the bone implant.

7. A method of securing a bone implant to a rod, the method comprising:
coupling a tulip assembly delivery tool to a tulip assembly, the tulip assembly including a tulip, a collet at least partially disposed within the tulip, and a saddle within the tulip and at least partially proximal to the collet;
using the tulip assembly delivery tool to advance the tulip assembly over a guide rod;
advancing the collet against a proximal end of a proximal region of a bone implant and causing a distal end of the collet to expand as it is advanced over the bone implant proximal region;
continuing to advance the collet over the bone implant proximal region until a radially inward protrusion of the collect moves radially inward into a recessed region of the bone implant, the recessed region of the bone implant having a proximal end with an undercut ledge configuration;
advancing the bone implant into bone; and
positioning a rod through a side opening in the tulip and screwing a set screw into the tulip until the set screw applies a stabilizing force on the rod and until the tulip is secured in place relative to the bone implant.

8. The method of claim 7, wherein the step of using the tulip assembly delivery tool to advance the tulip assembly over the guide rod further comprises coupling the guide rod to the proximal region of the bone implant such that the guide rod extends proximally from the bone implant.

9. The method of claim 8, wherein the step of coupling a guide rod to the proximal region of the bone implant further comprises engaging an internal thread of the implant proximal region with the guide rod.

10. The method of claim 7, wherein the bone implant is advanced into bone with a bone implant delivery tool that is coupled to a proximal region of the bone implant; and the method further comprises the step of disengaging the bone implant delivery tool from the bone implant and removing the bone implant delivery tool.

11. The method of claim 7, wherein the tulip assembly is modular and is adapted to be delivered separately from the bone implant and movably secured to the bone implant after the bone implant has been at least partially implanted within bone.

12. The method of claim 7, wherein the step of advancing the collet against a proximal end of a proximal region of a bone implant further comprises positioning a radially inward protruding implant interface region of the collet within a radially recessed collet receiving region of the bone implant.
